(19) United States
(12) Patent Application Publication
(10) Pub. No.: US 2017/0362283 A1
(43) Pub. Date: Dec. 21, 2017

(54) NISIN-BASED COMPOUNDS AND USE THEREOF IN THE TREATMENT OF BACTERIAL INFECTIONS

(71) Applicant: UNIVERSITEIT UTRECHT HOLDING B.V., Utrecht (NL)

(72) Inventors: Nathaniel Isaac MARTIN, Utrecht (NL); Timo KOOPMANS, Utrecht (NL); Thomas Melvin WOOD, Utrecht (NL); Laurens Henri Johan KLEIJN, Utrecht (NL)

(21) Appl. No.: 15/543,660

(22) PCT Filed: Jan. 15, 2016
(86) PCT No.: PCT/EP2016/050827

§ 371 Date: Jul. 14, 2017

(30) Foreign Application Priority Data
Jan. 19, 2015 (NL) 2014152
Apr. 20, 2015 (NL) 2014670

(51) Int. Cl. C07K 14/315; A61K 38/00

(52) U.S. Cl. CPC C07K 14/315; A61K 38/00

(57) ABSTRACT

The invention relates to new antimicrobial compounds derived from nisin. In particular, the compounds are based on the unsubstituted nisin [1-12] structure, wherein said compounds have an antimicrobial activity exceeding the activity of the unsubstituted nisin [1-12] structure.

### FIELD OF THE INVENTION

The invention relates to the field of medicine. More in particular, the invention relates to antimicrobial compounds based on nisin and the use thereof as medicaments.

### BACKGROUND OF THE INVENTION

Nisin is a polycyclic antibacterial peptide produced by the bacterium Lactococcus lactis. Because of its antibacterial activity it is often used as an additive in food, like processed cheese, meats, and milk. In its original form nisin has 34 amino acids, including the uncommon lanthionine (Lan), methyllanthionine (MeLan), didehydroalanine (Dha) and didehydroaminobutyric acid (Dhb) that are introduced during posttranslational modifications of the originating 57-aa precursor peptide. Nisin is a member of the class of molecules referred to as ‘lantibiotics’. Other members of this class are subtilin and epidermin. Nisin was already approved for use in food in the late 1960s. Its E number is E234. Because of its antibacterial properties it has also been envisioned as an antibiotic. However, one of the major disadvantages of using it as a medicinal antibiotic in humans is that it metabolizes relatively readily in the human stomach and in human blood.

Variations to nisin were reported in literature. WO 2007/103548 discloses a 12 amino acid containing structure herein further referred to as “nisin [1-12]” that is being connected through a linker to an antibiotic moiety, in particular to vancomycin. WO 2014/085637 describes a 5-ring nisin-based lantibiotic wherein some of the amino acids can be replaced and which can further comprise a hydrocarbon substituent. In WO 2010/058238 a 4-ring lantibiotic comprising a wide range of substituents, e.g. C₁-C₂₀ alkyl, is disclosed. Further nisin derivatives having at least one amino acid substituted in the peptide sequence were disclosed in WO 2009/13545. Most if not all of these known compounds are large and therefore degraded rapidly through the action of proteolytic enzymes. It goes without saying that there is a continuous need for new antibiotics that act against a wide variety of microbes, and that do not become degraded rapidly once present in circulation.

### SUMMARY OF THE INVENTION

The present invention pertains to an antimicrobial compound according to Formula (1),

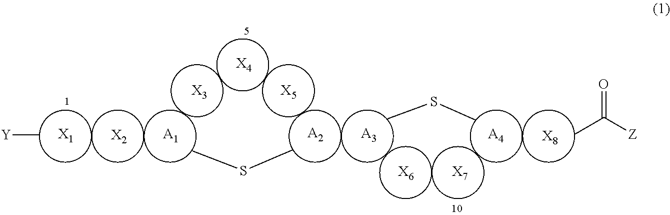

wherein:
Z is selected from any one of the substituents NHR₁, NR₁R₂, OR₁ and SR₁, and Y is selected from any one of the substituents NHR₃, NR₃R₄, NHCR₃R₄, NHCOR₃, NHCSR₃, NHOR₃ and NHC(NR₃NHR₄), wherein R₁, R₂, R₃ and/or R₄ is a substituted or non-substituted substituent selected from: alkyl, alkenyl, alkynyl, cycloalkyl, aryl, and polyaryl, wherein said substituent comprises at least 2, 4 or 6 carbon atoms and at most 30, 40 or 50 carbon atoms;
A₁ and A₃ are independently D-Alanine or D-Aminobutyric acid;
A₂ and A₄ are L-alanine;
A₁+A₂ and A₃+A₄ independently form a (2S,6R)-lanthionine or (2S,3S,6R)-methyllanthionine linkage; and
X₁ to X₈ are each independently selected from natural or non-natural amino acids.

In a preferred embodiment, Y and Z each have a molecular weight of less than 1200 Dalton. In a further preferred embodiment, and in relation to all different antimicrobial compounds as disclosed herein, R₁, R₂, R₃ and R₄ are each a substituted or unsubstituted substituent, independently selected from: C₄ to C₅₀ alkyl, C₂ to C₅₀ alkenyl, C₂ to C₅₀ alkynyl, cycloalkyl, aryl and polyaryl. In another preferred embodiment, both R₁ and R₂ and/or both R₃ and R₄ are substituted or non-substituted substituents selected from: C₅ to C₄₀ alkyl, C₄ to C₄₀ alkenyl, C₄ to C₄₀ alkynyl, cycloalkyl, aryl and polyaryl.

In another preferred embodiment, X₈ is an amino acid that carries no charge on the side-chain. More preferably, X₈ is lysine which is acylated on the side-chain. Even more preferably, X₈ is lysine which is acetylated on the side-chain. In a highly preferred aspect of the invention the structures of the present invention have an antimicrobial activity exceeding the activity of the unsubstituted nisin [1-12] structure known in the art.

### DETAILED DESCRIPTION

The objective of the present invention is to provide novel antimicrobial compounds. The nisin-derived compounds of the present invention exhibit antimicrobial activity, in particular antibacterial activity. Moreover, the compounds of the present invention generally are capable of killing drug-resistant strains, in particular drug-resistant strains of Gram-positive bacteria. The inventors have surprisingly found that the bactericidal mechanism is different from that of nisin. The compounds of the invention are capable to bind to the pyrophosphate of lipid II in the bacterial cell wall, similar to nisin. Nisin additionally causes pores to form in the cell wall, whereas the compounds of the present invention do not cause such pore formation. It is further noted that the nisin [1-12] structure known in the art, wherein Z is OH (R₁ is hydrogen), and Y is NH₂ (R₃ and R₄ are hydrogen), generally does not have significant antimicrobial activity. Therefore, it was surprising that the compound of the present invention exhibits considerable antimicrobial activity, and appeared generally active against a wide variety of bacterial strains. A further advantage appeared to be the improved stability of the compounds of the present invention in human serum compared to nisin.

The present invention relates to an antimicrobial compound according to Formula (1),

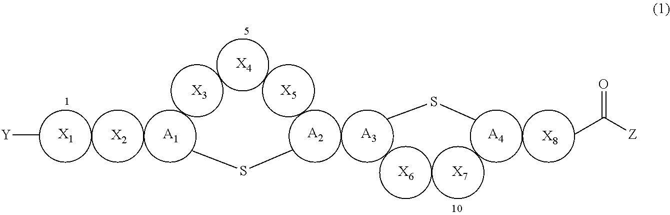

wherein Z is selected from any one of the substituents NHR₁, NR₁R₂, OR₁ and SR₁, and Y is selected from any one of the substituents NHR₃, NR₃R₄, NHCR₃R₄, NHCOR₃, NHCSR₃, NHOR₃ and NHC(NR₃NHR₄), wherein R₁, R₂, R₃ and/or R₄ is a substituted or non-substituted substituent selected from: alkyl, alkenyl, alkynyl, cycloalkyl, aryl, and polyaryl, wherein said substituent comprises at least 2, 4 or 6 carbon atoms and at most 30, 40 or 50 carbon atoms; A₁ and A₃ are independently D-Alanine or D-Aminobutyric acid; A₂ and A₄ are L-alanine; A₁+A₂ and A₃+A₄ independently form a (2S,6R)-lanthionine or (2S,3S,6R)-methyllanthionine linkage; and X₁ to X₈ are each independently selected from natural or non-natural amino acids.

In a preferred embodiment, Y and Z each have a molecular weight of less than 1200 Dalton. In another preferred embodiment, R₁, R₂, R₃ and/or R₄ is a substituted or unsubstituted substituent, independently selected from: C₄ to C₅₀ alkyl, C₂ to C₅₀ alkenyl, C₂ to C₅₀ alkynyl, cycloalkyl, aryl and polyaryl. In an especially preferred embodiment, both R₁ and R₂ and/or both R₃ and R₄ are substituted or non-substituted substituents selected from: C₅ to C₄₀ alkyl, C₄ to C₄₀ alkenyl, C₄ to C₄₀ alkynyl, cycloalkyl, aryl and polyaryl. In a further preferred embodiment, X₈ is an amino acid that carries no charge on the side-chain, preferably lysine that is acylated or acetylated on the side-chain. Preferably, it is acetylated.

In a particular aspect the invention relates to an antimicrobial compound according to the invention, wherein Z has a molecular weight of less than 1000 Dalton, preferably less than 800 Dalton, and more preferably less than 600 Dalton; and/or Y has a molecular weight of less than 1000 Dalton, preferably less than 800 Dalton, and more preferably less than 600 Dalton. In one particular aspect, Y is not NH₂, and Z is not OH or NH—CH₃, because it was found that a compound according to Formula (1) with these Y and Z groups did not exceed the antimicrobial activity of the unsubstituted nisin [1-12] structure. Hence, in a highly preferred aspect, the invention relates to an antimicrobial compound according to the present invention, wherein said compound has an antimicrobial activity exceeding the activity of the unsubstituted nisin [1-12] structure. Preferably, the amide-form of Z independently lacks antimicrobial activity.

In another preferred aspect, the compound of the present invention exhibits an MIC value below 100 μg/ml, preferably below 70 μg/ml, 50 μg/ml, 20 μg/ml, and most preferably below 10 μg/ml.

The invention furthermore relates to an antimicrobial compound according to the invention, for use in the treatment of a bacterial infection. The invention also relates to a pharmaceutical composition comprising the antimicrobial compound according to the invention, and a pharmaceutically acceptable diluent and/or carrier.

The invention furthermore relates to a use of an antimicrobial compound according to the invention for the manufacture of a medicament for use in the treatment of an infection, preferably a bacterial infection.

In yet another embodiment, the invention relates to a method of treating a subject suffering from a bacterial infection, comprising administering an antimicrobial compound according to the invention, or a pharmaceutical composition according to the invention, to said subject.

Preferred compounds as disclosed herein are compounds (6), (10), (12) and (20). Especially preferred are compounds (10), (12) and (20). Also preferred is compound (24) carrying R₁ structure (e) as disclosed herein. A very highly preferred compound is compound (12).

Preferred amino acids used in the compounds of the invention are those derived from known type A lantibiotics, in particular nisin, subtilin, gallidermin and epidermin. Specific compounds of the invention and their amino acid sequences are exemplified in Table 1. In a preferred embodiment of the invention, the compound according to Formula (1) wherein X₆ is proline and X₇ is glycine, and A₃ is D-aminobutyric acid and A₄ is L-alanine.

In a preferred embodiment of the invention, in the compound according to Formula (1), X₆ is proline, X₇ is glycine, A₃ is D-aminobutyric acid and A₄ is L-alanine. The remaining amino acids can be any known natural or non-natural amino acid. They can be incorporated by general methods known to the person skilled in the art. An example of such a method can for instance be gleaned in Rink et al. (in Appl. Environ. Microbiol., September 2007, pp. 5809-5816).

TABLE 1
Examples of X and A amino acid residues within the nisin-derived compounds according
to the present invention. The left column shows the lantibiotics from which the indicated
amino acids (given here with their respective 3-letter notation) are chosen.

| | X₁ | X₂ | X₃ | X₄ | X₅ | X₆ | X₇ | X₈ | A₁ | A₂ | A₃ | A₄ |
|---|---|---|---|---|---|---|---|---|---|---|---|---|
| Nisin | Ile | Dhb | Ile | Dha | Leu | Pro | Gly | Lys | D-Ala | L-Ala | D-Abu | L-Ala |
| Subtilin | Trp | Lys | Glu | Dha | Leu | Pro | Gly | Val | D-Ala | L-Ala | D-Abu | L-Ala |
| Subtilin | Trp | Lys | Glu | Dha | Leu | Pro | Gly | Ala | D-Ala | L-Ala | D-Abu | L-Ala |
| Gallidermin | Ile | Ala | Lys | Phe | Leu | Pro | Gly | Ala-Lys | D-Ala | L-Ala | D-Abu | L-Ala |
| Epidermin | Ile | Ala | Lys | Phe | Ile | Pro | Gly | Ala-Lys | D-Ala | L-Ala | D-Abu | L-Ala |

In one particular embodiment, the invention relates to a nisin-derived antimicrobial compound having a Minimum Inhibitory Concentration (MIC) value below 100 μg/ml. Preferably, the compound has a MIC value below 70 μg/ml, more preferably below 50 μg/ml, even more preferably below 20 μg/ml, and most preferably below 10 μg/ml. Determining the MIC value is a standard technique well known to the skilled person, in particular the MIC value can be measured using method M07-A9 (CLSI standard, January 2012, Vol. 32, No. 2, “Methods for Dilution Antimicrobial Susceptibility Tests for Bacteria That Grow Aerobically”).

The compound of the invention comprises a Y and a Z substituent which may be the same or different. The precursor of the substituent Z and/or Y, preferably Z, of the inventive compounds of Formula (1) generally lack antimicrobial activity themselves, which means that Z taken individually lacks antimicrobial activity. Examples of such precursors of the Z substituent include H₂NR₁, HNR₁R₂, HOR₁ and HSR₁. Examples of such precursors for the Y substituent include R₃HCO R₃R₄CO, R₃COOH, R₃CSOH and R₃—I. The precursors can generally be used in the process of preparing the antimicrobial compound of the invention. The term “lack antimicrobial activity” means that no relevant antimicrobial or antibacterial activity could be measured using conventional techniques known in the art. The substituent is thus not another antibacterial compound or a derivative thereof. The antimicrobial compounds disclosed in WO 2007/103548 (e.g. Z being vancomycin) are not considered to be part of an antimicrobial compound according to the present invention.

The compounds according to the present invention comprise a substituent Z having a molecular weight of less than 1200 Dalton. Preferably, the molecular weight is less than 1000 Dalton, more preferably less than 800 Dalton, and most preferably less than 600 Dalton. The compound of the invention further comprises a substituent Y having a molecular weight of less than 1200 Dalton. Preferably, the molecular weight is less than 1000 Dalton, more preferably less than 800 Dalton, and most preferably less than 600 Dalton. An advantage of such relatively small substituents is that the preparation of the compounds of the invention is relatively simple and can be kept economically and commercially interesting.

The antimicrobial compound of the present invention is based on the original nisin [1-12] structure and preferably comprises a substituent Y (having an R₃ and/or R₄) and/or Z, preferably Z, (having an R₁ and/or R₂), which is a substituted or unsubstituted substituent independently selected from C₄ to C₅₀ alkyl, C₂ to C₅₀ alkenyl, C₂ to C₅₀ alkynyl, cycloalkyl, aryl and polyaryl. In the context of the present invention the wording “substituted” refers to the substitution of the substituent with a further substituent and/or the modification of the substituent with a hetero atom like O, S, or N for example. Preferably, both R₁ and R₂, and/or both R₃ and R₄, even more preferably both R₁ and R₂, are independently substituted or non-substituted substituents selected from C₅ to C₄₀ alkyl, C₄ to C₄₀ alkenyl, C₄ to C₄₀ alkynyl, cycloalkyl, aryl and polyaryl. Even more preferred are substituted or non-substituted substituents selected from alkyl, alkenyl, alkynyl, cycloalkyl, aryl or polyaryl, wherein the substituent comprises at least 4 carbon atoms, more preferably at least 5 carbon atoms and most preferably at least 6 carbon atoms, and at most 50 carbon atoms, more preferably at most 40 carbon atoms and most preferably at most 30 carbon atoms.

The inventors have found that, as an exception to the other compounds of the present invention, a compound according to Formula (1) wherein Y is NH₂, and Z is OH or NH—CH₃ does not provide antimicrobial activity exceeding the antimicrobial activity of the nisin [1-12] structure.

In a preferred aspect of the present invention, the antimicrobial compounds according to the invention are as shown in Table 2 and according to basic Formula (1) as given above, wherein X₁=Ile, X₂=Dhb, X₃=Ile, X₄=Dha, X₅=Leu, X₆=Pro, X₇=Gly; A, =D-Ala, A₂=L-Ala, A₃=D-Abu and A₄=L-Ala, wherein Z is a substituent of the type NHR₁, wherein Y is a substituent of the type NHR₃, and wherein X₈, R₁ and R₃ are selected from the structures as provided in Table 2, leading to compounds referred to as Formulas (2) to (23).

TABLE 2
Preferred antimicrobial compounds with Formulas (2) to (23)-as indicated in the
right column-based on Formula (1) with different X₈, R₁ and R₃ groups as indicated.

| X₈ | R₁ | R₃ | |
|---|---|---|---|
| Lys | —C₆H₁₃ | H | (2) |
| Lys | —C₇H₁₅ | H | (3) |
| Lys | —C₈H₁₇ | H | (4) |
| Lys | —C₉H₁₉ | H | (5) |
| Lys | —C₁₀H₂₁ | H | (6) |
| Lys | —C₁₁H₂₃ | H | (7) |
| Lys | —C₁₂H₂₅ | H | (8) |
| Lys | —C₁₃H₂₇ | H | (9) |
| Lys | —C₁₄H₂₉ | H | (10) |
| Lys | —C₁₅H₃₁ | H | (11) |
| Lys | 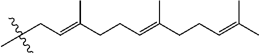 [SMILES: *C/C=C(\C)CC/C=C(\C)CCC=C(C)C] | | (12) |
| Lys | 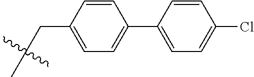 [SMILES: *Cc1ccc(-c2ccc(Cl)cc2)cc1] | H | (13) |
| Lys | 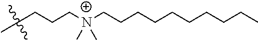 [SMILES: *CCC[N+](C)(C)CCCCCCCCCC] | H | (14) |
| Lys | 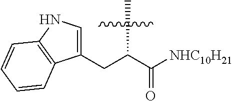 | H | (15) |
| Lys | 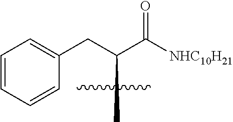 | H | (16) |
| Lys | 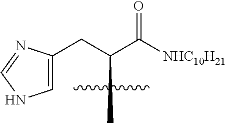 | H | (17) |
| Lys | 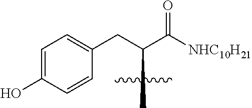 | H | (18) |
| Lys((C═O)C₈H₁₇) | —CH₃ | H | (19) |
| Acetyllysine | —C₁₂H₂₅ | H | (20) |
| 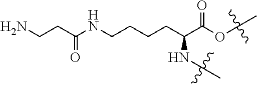 [SMILES: *N[C@@H](CCCCNC(=O)CCN)C(=O)O*] | —C₁₂H₂₅ | H | (21) |
| 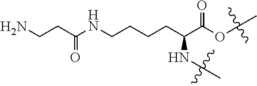 [SMILES: *N[C@@H](CCCCNC(=O)CCN)C(=O)O*] | —C₁₂H₂₅ | 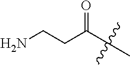 [SMILES: *C(=O)CCN] | (22) |
| Lys | 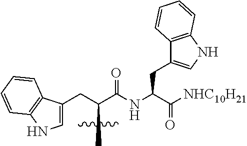 | H | (23) |

In yet another preferred embodiment, the Z substituent comprises an R₁ group selected from the ones indicated in Table 2, and the Y substituent is NHR₃ wherein R₃ is hydrogen.

A precursor to the antimicrobial compounds in accordance with the invention is comparative compound E; according to Formula (1) wherein R₁ has the following structure (a):

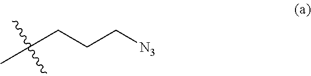 [SMILES: *CCCN=[N+]=[N-]]

The invention also relates to an antimicrobial compound according to Formula (24),

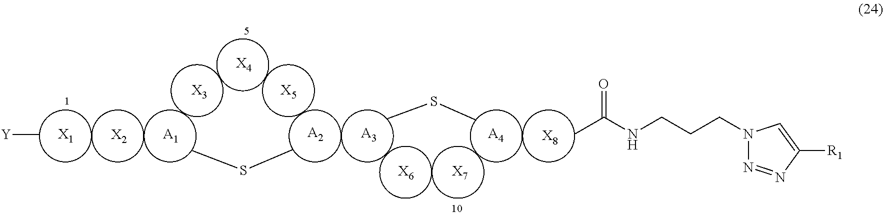

wherein:

Y is selected from any one of the substituents NHR₃, NR₃R₄, NHCOR₃, NHCSR₃, NHOR₃ and NHC(NR₃NHR₄), wherein R₁, R₃ and R₄ are independently selected substituents as disclosed herein; A₁, A₃ are independently D-Alanine or D-Aminobutyric acid; A₂ and A₄ are L-alanine; A₁+A₂ and A₃+A₄ independently form a (2S,6R)-lanthionine or (2S,3S,6R)-methyllanthionine linkage; and X₁ to X₈ are each independently selected from natural or non-natural amino acids.

In a preferred embodiment, Y and R₁ each have a molecular weight of less than 1200 Dalton. In another preferred embodiment, X₈ is an amino acid that carries no charge on the side-chain. More preferably, X₈ is lysine which is acylated on the side-chain, even more preferably, X₈ is lysine which is acetylated on the side-chain.

The invention further pertains to the antimicrobial compounds according to Formula (24) comprising an R₁ group selected from the following four structures (b), (c), (d), and (e) (see also Table 4 below):

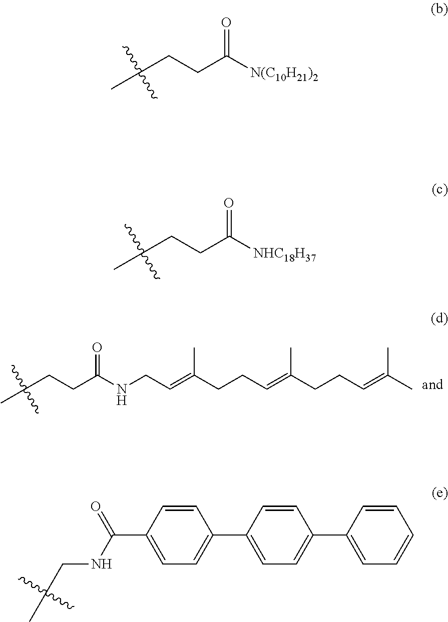

In one preferred embodiment, the present invention relates to an antimicrobial compound according to Formula (24), wherein the R₁ group is selected from the four structures (b), (c), (d) and (e), and the Y substituent is NHR₃ wherein R₃ is hydrogen. In a further preferred embodiment, the present invention relates to an antimicrobial compound according to Formula (24), wherein the R₁ group is structure (e), and the Y substituent is NH₂.

The antimicrobial compounds of the present invention according to Formulae (1) to (24), with their indicated R groups, preferably all exhibit antimicrobial activity exceeding the activity of the unsubstituted nisin [1-12] structure. Especially preferred antimicrobial compounds of the present invention are those according to Formulas (6), (7), (8), (9), (10), (12), (13), (14), (15), (16), (17), (18), (20) and the compound according to Formula (24) carrying structure (e) as the R1 group. These compounds particularly provide for an even higher antimicrobial activity and/or stability in human serum and/or lower hemolytic activity than the other compounds of the invention. Highly preferred is an antimicrobial compound according to Formula (12).

The compounds of the invention can be prepared by starting with the basic nisin [1-12] structure which is then substituted at the C-terminus side by coupling with a nucleophile precursor or an alkyne precursor at the Z substituent forming a covalent connection. The basic nisin [1-12] structure may be prepared by treating nisin with an enzyme capable of cutting nisin at position 12. An example of such an enzyme is Trypsin. Materials and methods are provided in the accompanying examples.

The present invention further pertains to a combination of an antimicrobial compound of the invention and an active pharmaceutical ingredient. The active pharmaceutical compound can be any such compound known to the skilled person. Preferably, the active pharmaceutical ingredient is a second antimicrobial agent. In the context of the present application the term “combination” refers to a composition comprising both the antimicrobial compound of the invention and an active pharmaceutical ingredient, or to a plurality of pharmaceutical compositions comprising both the antimicrobial compound and the pharmaceutical ingredient in two or more different compositions. The present invention therefore also pertains to a kit-of-parts comprising an antimicrobial compound and an active pharmaceutical ingredient, in particular a pharmaceutical ingredient being a second antimicrobial agent. The plurality of compositions of the invention may be administered to a patient simultaneously and/or consecutively.

Examples of such antimicrobial agents include aminoglycosides such as amikacin, gentamicin, kanamycin, neomycin, netilmicin, tobramycin, paromomycin, streptomycin and spectinomycin; ansamycins like rifaximin, geldanamycin and herbimycin; carbapenems such as ertapenem, doripenem and meropenem; cephalosporins like cefadroxil, cefazolin, cefalotin, cefaclor, cefamandole, cefoxitin, cefprozil, cefuroxime, cefixime, cefdinir, cefditoren, cefoperazone, cefotaxime, cefpodoxime, ceftazidime, cefibuten, ceftizoxime, ceftriaxone, cefepime, ceftaroline fosamil and ceftobiprole; glycopeptides such as teicoplanin, vancomycin, oritavancin, telavancin, dalbavancin and ramoplanin; lincosamides like clindamycin and lincomycin; lipopeptides such as daptomycin; macrolides like azithromycin, clarithromycin, dirithromycin, erythromycin, roxithromycin, troleandomycin, telithromycin, and spiramycin; monobactams such as aztreonam; nitrofurans like furazolidone, nifrofurantoin; oxazolidinones such as linezolid, posizolid, radezolid and torezolid; penicillins such as amoxicillin, ampicillin, aziocillin, carbenicillin, cloxacillin, dicloxacillin, flucloxacillin, meziocillin, methicillin, nafcillin, oxacillin, penicillin G, penicillin V, piperacillin, temocillin, ticarcillin; penicillin combinations such as amoxicillin/clavulanate, ampicillin/sulbactam, piperacillin/tazobactam and ticarcillin/clavulanate; polypeptides such as bacitracin, colistin and polymyxin B; quinolones such as ciprofloxacin, enoxacin, gatifloxacin, gemifloxacin, levofloxacin, levofloxacin, lomefloxacin, moxifloxacin, nilidixic acid, norfloxacin, trovafloxacin, grepafloxacin, sparfloxacin and temafloxacin; sulfonamides like mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (co-trimoxazole), sulfonamidochrysoidine; tetracyclines like demeclocycline, doxycycline, minocycline, oxytetracycline, tetracycline; drugs against mycobacteria such as ciofazimine, dapsone, capreomycin, cycloserine, ethambuto, ethionamide, isoniazid, pyrazinamide, rifampcin, rifabutin, rifapentine and streptomycin; and arsphenamine, chloramphenicol, fosfomycin, fusidic acid, metronidazole, metronidazole, mupirocin, platensimycin, quinupristin/dalfopristin, thiamphenicol, tigecycline, tinidazole and trimethoprim.

In one embodiment of the invention, the molar ratio between the antimicrobial compound and the active pharmaceutical ingredient is at most 10:1, preferably at most 5:1, and most preferably at most 2:1, and generally at least 1:20, preferably at least 1:10, more preferably at least 1:5, and most preferably at least 1:2.

The invention further pertains to a pharmaceutical composition comprising the antimicrobial compound or the combination of the invention, and a pharmaceutically acceptable diluent or carrier. The term “pharmaceutical composition” or “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable diluent or carrier” refers to an ingredient in a pharmaceutical formulation, other than an active ingredient, which is nontoxic to a subject. A pharmaceutically acceptable diluent or carrier includes, but is not limited to, water, a buffer, excipient, stabilizer, or preservative.

In another embodiment of the invention, the antimicrobial compound or the combination of the invention is divided over two or more pharmaceutical compositions, wherein the antimicrobial compound of the invention is comprised in one pharmaceutical composition and the active pharmaceutical ingredient is comprised in a second pharmaceutical composition. In this way the antimicrobial compound and the active pharmaceutical ingredient can be administered to a patient consecutively. It is also envisaged to provide compositions comprising part of the total amount of the antimicrobial compound and/or the active pharmaceutical ingredient.

In one embodiment, the invention pertains to the use of the antimicrobial compound, the combination of the invention or the pharmaceutical composition of the invention as a medicament. In yet another embodiment, the invention pertains to the use of the antimicrobial compound, the combination of the invention or the pharmaceutical composition of the invention in the treatment of infections, preferably bacterial infections.

The term “infection” as used herein refers to diseases caused by microorganisms, such as bacteria or a virus, to which the human or animal body reacts, generally causing an inflammatory reaction. The antimicrobial compounds of the present invention are particularly effective against bacteria. Such bacteria may be Gram-negative and Gram-positive bacteria. Of particular interest are bacterial strains which comprise a cell wall of which the precursor is lipid II. Examples of Gram-negative bacteria include Coccobacilli such as Hemophilus influenzae, B. pertussis, Brucella spp., F. tularensis, P. multocida, and Legionella pneumophila; Cocci such as Neisseria gonorrhoeae, Neisseria meningitidis and Moraxella catarrhalis; Bacilli like Klebsiella pneumoniae, Pseudomonos aeruginosa, Proteus mirabilis, Enterobacter cloacae, Heliobacter pylori, Serratia marcescens, Salmonella enteritidis, Salmonella typhi; and Acinetobacter baumannii. Examples of Gram-positive bacteria include Staphylococcus like Staphylococcus aureus, Staphylococcus epidermidis and Staphylococcus saprophyticus; Streptococcus such as Streptococcus pyogenes, Streptococcus agalactiae, Streptococcus pneumoniae, Viridans mutans, Enterococcus faecalis, and Enterococcus faecium; Micrococcaceae such as Micrococcus luteus; Corynebacterium, Mycobacterium, Firmicutes, Streptomyces, Clostridium, Listeria and Bacillus.

The antimicrobial compounds of the invention are generally active against drug-resistant bacteria. The invention therefore also pertains to the use of the antimicrobial compounds in the treatment of drug-resistant bacteria. With the wording “drug-resistant” it is meant that a resistance towards one or more existing drugs exists. Additionally, pharmaceutical compositions and the combinations comprising the antimicrobial compound of the invention can also be used in the treatment of drug-resistant bacteria.

In one embodiment, the drug-resistant bacteria are resistant to at least one drug selected from the group consisting of penicillin, beta-lactam, vancomycin, linezolid, fluoroquinolone, clindamycin, carbapenem, isoniazid, rifampin, tetracycline, cyphalosporin, aminoglycoside, methicillin, ampicillin and daptomycin. Examples of the drug-resistant bacteria include methicillin-resitant Staphylococcus aureus (MRSA), vancomycin-resistant Staphylococcus aureus (VRSA), penecillin-resistant Streptococcus pyogenes, macrolide-resistant Streptococcus pyogenes, penicillin-resistant Streptococcus pneumonia, beta-lactam-resistant Streptococcus pneumonia, penicillin-resistant Enterococcus faecalis, vancomycin-resistant Enterococcus faecalis, linezolid-resistant Enterococcus faecalis, penicillin-resistant Enterococcus faecium, vancomycin-resistant Enterococcus faecium, linezolid-resistant Enterococcus faecium, Pseudomonas aeruginosa, fluoroquinolone-resistant Clostridium difficile, clindamycin-resistant Clostridium difficile, fluoroquinolone-resistant Escherichia coli, Salmonella, Acinetobacter baumannii (MRAB), carbapenem-resistant Klebsiella pneumoniae, Mycobacterium tuberculosis (XDR TB), isoniazid-resistant Mycobacterium tuberculosis, rifampin-resistant Mycobacterium tuberculosis, tetracycline-resistant Neisseria gonorrhoeae, aminoglycoside-resistant Neisseria gonorrhoeae, cephalosporin-resistant Neisseria gonorrhoeae, and penicillin-resistant Neisseria gonorrhoeae.

Specific examples of drug-resistant bacteria include Streptococcus mutans (ATCC 700610), Streptococcus mutans (strain Xc), Streptococcus sobrinus (ATCC 33478), Streptococcus uberis (strain 1978), Streptococcus uberis (strain 1979), Streptococcus uberis (strain 1980), Streptococcus uberis (strain 1981), Streptococcus pyogenes (strain 5448), Streptococcus pyogenes (strain JRS4), Streptococcus pyogenes (ATCC BAA-595), Streptococcus. Pneumoniae, Streptococcus mitis, Streptococcus sanguis, Streptococcus bovis, Streptococcus salivarius, Streptococcus intermedius, Streptococcus viridans, Streptococcus oralis, Streptococcus salivarus, Staphylococcus lugdunensis, Staphylococcus aureus (ATCC BAA-1717), Staphylococcus aureus (ATCC 25904), Staphylococcus aureus (strain MRSA-16), Staphylococcus aureus (strain Cowan), Staphylococcus capiticus (strain V19), Staphylococcus epidermidis (strain 1587), Staphylococcus hominis (strain V27), Staphylococcus warneri (strain V64), Staphylococcus saprofyticus (strain NCTC 7292), Staphylococcus haemolyticus (strain V8/1), Salmonella typhimurium, Eneterococcus faecium (ATCC 700221), Eneterococcus faecium (daptomycin resistant strain), Eneterococcus faecium (linezolid resistant strain), Eneterococcus faecium (ampicillin resistant strain), Enterococcus faecium (vancomycin-resistant strains E0013, E0072, E0300, E0321, E0333, E0338, E0341, E0506, E0745, E1130, E1441, E1679, E1763, E2297, E2359, E2365, E2373, E6016, E7312, E7314, E7319, E7329, E7401, E7403, E7413, E7424, E7464, E8218, E8235, E8237), Eneterococcus faecalis (ATCC 700802), Eneterococcus faecalis (strain JH2-2), Eneterococcus faecalis (strain MMH594), Eneterococcus faecalis (ATCC 29212), Eneterococcus faecalis (ATCC 47077), Eneterococcus hirae, Eneterococcus casseliflavus, Eneterococcus gallinarum, Eneterococcus. Raffinosus, Eneterococcus avium, Eneterococcus cecorum, Eneterococcus saccharominimus, Eneterococcus columbae, Eneterococcus durans, Klebsiellsa pneumoniae, Lactobacillus paracasei, Clostridium tetani, Clostridium botulinum, Clostridium perfringes, Clostridium difficile, Bacillus anthracis and Listeria moncytogenes. The above drug-resistant strains were identified and known at the Utrecht Medical Center.

As used herein, “treatment” (and grammatical variations thereof such as “treat” or “treating”) refers to clinical intervention in an attempt to alter the natural course of the individual or animal being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

An “effective amount” of an agent, e.g., a pharmaceutical formulation, refers to an amount effective, at dosages and for periods of time necessary, to achieve the desired therapeutic or prophylactic result.

“Natural or non-natural amino acids” refers to any of the common naturally occurring amino acids as well as modified, derivatized, enantiomeric, rare and/or unusual amino acids which may be synthetically obtained or originating from a natural source. Examples of naturally occurring amino acids includes alanine (Ala, A), cysteine (Cys, C), aspartic acid (Asp, D), glutamic acid (Glu, E), phenylalanine (Phe, F), glycine (Gly, G), histidine (His, H), isoleucine (Ile, I), lysine (Lys, K), leucine (Leu, L), methionine (Met, M), asparagine (Asn, N), proline (Pro, P), glutamine (Gln, Q), arginine (Arg, R), selenocysteine (Sec, U), serine (Ser, S), threonine (Thr, T), valine (Val, V), tryptophan (Trp, W), and tyrosine (Tyr, Y). Examples of the modified amino acids include hydroxyproline, hydroxylysine, actetyllysine, desmosine, isodesmosine, ε-N-methyllysine, ε-N-trimethyllysine, methylhistidine, dehydrobutyrine (Dhb), dehydroalanine (Dha), α-aminobutyric acid (Abu), 2,3-diaminopropioninc acid, β-alanine, γ-aminobutyric acid, homocysteine, homoserine, citrulline and ornithine.

The invention is exemplified in the following non-limiting examples.

### EXAMPLES

### Example 1. Preparation of Nisin-Derived Antimicrobial Compounds

All reagents employed were of American Chemical Society (ACS) grade or finer and were used without further purification unless otherwise stated. Flash chromatography was performed using Merck type 60, 230-400 mesh silica gel. Peptides were purified on a Reprospher 100 C8-Aqua column (10 250×20 mm) at a flow rate of 12 mL·min⁻¹. High resolution mass spectrometry (HRMS) analysis was performed using an ESI-TOF LC/MS instrument ¹H NMR spectra were recorded at 400 MHz with chemical shifts reported in parts per million (ppm) downfield relative to tetramethylsilane (TMS). ¹H NMR data are reported in the following order: multiplicity (s, single; d, doublet; t, triplet, q, quartet; qn, quintet and m, multiplet), number of protons, coupling constant (J) in Hertz (Hz). When appropriate, the multiplicity is preceded by br, indicating that the signal was broad. ¹³C NMR spectra were recorded at 100 MHz with chemical shifts reported relative to the residual carbon resonance of the solvent used. All literature compounds had ¹H NMR, and mass spectra consistent with the assigned structures.

### Preparation of the Nisin [1-12] Structure (Comparative Compound A)

Nisin (600 mg, 0.18 mmol) was dissolved in 250 mL Tris buffer (25 mmol NaOAc, 5 mmol Tris acetate, 5 mmol CaCl₂, pH 7) and the solution was cooled on ice for 15 min. Trypsin (50 mg) was added and the mixture was stirred at RT for 15 min. The mixture was then heated to 30° C. for 16 hours and an aliquot was analyzed by HPLC. Another 50 mg of trypsin was added and after an additional 24 hours the reaction was complete, as evidenced by HPLC. The reaction was acidified with HCl (1 N) to a pH of 4 and solvents were removed in vacuo. The nisin [1-12] structure was isolated from the mixture by preparative HPLC. Product fractions were lyophilized to obtain a white powder (80 mg, 39%).

### Preparation of the Farnesyl-Amine (According to G M Coppola and M Prashad, Synthetic Communications 23, No. 4 (1993): 535-41)

Lithium bis(trimethylsilyl) amide (7.7 mL; 1.0 M in THF) was added to trans,trans-farnesyl bromide (6.7 mmol, 1.9 g) under a blanket of argon and the mixture was stirred for 16 hours, followed by quenching with a saturated ammonium chloride solution. The mixture was extracted twice with MTBE and the organic phases were combined and dried over Na₂SO₄. To this oil was added 31 mL MeOH and 4 mL CH₂Cl₂ and the resulting solution was stirred at room temperature for 16 hours. Solvents were removed under vacuum to give a brown solid as product (1.5 g, quant.).

¹H NMR (400 MHz, CDCl₃): δ 5.28-5.24 (m, 1H), 5.12-5.07 (m, 2H), 3.29-3.27 (d, 2H, J=7.0 Hz), 2.10-1.95 (m, 8H), 1.67-1.59 (m, 12H), 1.12 (s, 2H); ¹³C NMR (100 MHz, CDCl₃): δ□137.8, 135.2, 131.3, 125.4, 124.3, 123.9, 39.7, 39.5, 26.7, 26.4, 25.7, 17.6, 16.1, 16.0.

### Preparation of 2,5-dioxopyrrolidin-1-yl pent-4-ynoate

4-Pentynoic acid (2.00 g, 20.36 mmol) was dissolved in DMF (40 mL) and EDCl (5.84 g, 29.84 mmol, 1.5 equiv.) and NHS (4.68 g, 40.76 mmol, 2.0 equiv.) were added. The mixture was stirred for 16 hours at RT. After evaporation of DMF the residue was diluted in EtOAc (120 mL) and washed two times with NH₄Cl (1 M, 120 mL) and two times with saturated NaHCO₃ (120 mL). The organic layer was dried with Na₂SO₄ and the product was purified with flash column chromatography (EtOAc: PE) to obtain the desired activated ester as a white powder (3.3 g, 83%).

¹H NMR (400 MHz, CDCl₃): δ 2.89-2.83 (m, 4H), 2.63-2.59 (m, 4H), 1.55 (bs, 1H); ¹³C NMR (100 MHz, CDCl₃): δ 168.8, 167.0, 80.8, 70.0, 30.3, 25.5, 14.1.

### Preparation of Didecyl-Alkyne

Prepared via procedure 3 (p3) using didecylamine.

Yield: 105 mg, 30%

¹H NMR (400 MHz, CDCl₃): δ 3.31-3.27 (m, 2H), 3.22-3.18 (m, 2H), 2.54 (m, 4H), 1.95 (2, 1H), 1.56-1.48 (m, 4H), 1.27-1.25 (m, 28H), 0.90-0.85 (m, 6H); ¹³C NMR (100 MHz, CDCl₃): δ 170.0, 83.8, 68.5, 47.8, 46.1, 32.1, 31.9, 29.6, 29.5, 29.3, 22.7, 14.6, 14.1; HRMS calculated for C₂₅H₄₈NO [M+H]⁺: 378.3736, found 378.3743.

### Preparation of Octadecyl-Alkyne

Prepared via procedure 3 (p3) using octadecylamine.

Yield: 560 mg, 52%

¹H NMR (400 MHz, CDCl₃): δ 5.61 (bs, 1H), 3.27-3.21 (m, 2H), 2.53-2.50 (m, 2H), 2.39-2.34 (m, 2H), 1.99-1.97 (m, 1H), 1.47-1.47 (m, 3H), 1.27-1.24 (m, 32H), 0.88-0.84 (m, 3H); ¹³C NMR (100 MHz, CDCl₃): δ 170.7, 83.0, 69.2, 39.6, 35.4, 31.9, 29.7, 29.6, 29.5, 29.3, 26.9, 22.7, 15.0, 14.1; HRMS calculated for C₂₃H₄₄NO [M+H]⁺: 350.3423, found 350.3430.

### Preparation of Farnesyl-Alkyne

Prepared via procedure 3 (p3) using farnesyl-amine.

Yield: 540 mg, 60%

¹H NMR (400 MHz, CDCl₃): δ 5.69 (bs, 1H), 5.19-5.15 (m, 1H), 5.06-5.04 (m, 2H), 4.12-4.08 (m, 2H), 3.85-3.82 (m, 2H), 2.78 (s, 1H), 2.50-2.48 (m, 4H), 2.38-2.34 (m, 4H), 1.64 (s, 9H), 1.57 (s, 3H), 1.25-1.21 (m, 2H); ¹³C NMR (100 MHz, CDCl₃): δ 170.5, 140.2, 135.4, 131.4, 124.2, 123.7, 119.7, 83.0, 69.2, 39.7, 39.5, 37.6, 35.4, 26.7, 26.3, 25.7, 17.7, 16.3, 16.0, 14.9; HRMS calculated for C₂₀H₃₂NO [M+H]⁺: 302.2484, found 302.2474.

### Preparation of Terphenyl-Alkyne

A mixture of terphenyl carboxylic acid (250 mg, 1.0 equiv.) and thionyl chloride (10 mL, per mmol carboxylic acid) was refluxed until all solid was dissolved followed by additional heating for 16 hours. After evaporation of excess thionyl chloride at reduced pressure the obtained acid chloride was dried in vacuo. The acid chloride was dissolved in 15 mL DCM and propargylamine HCL (183 mg, 2.0 equiv.) was added. Upon addition of TEA (558 μL, 4.0 equiv.) the solution turned into a thick white suspension. 5 mL of DCM was added to facilitate stirring. TLC showed little conversion after 3 hours. Pyridine (790 μL, 10 equiv.) was added and the mixture was heated to reflux. After 2 hours the reaction was complete. Solvents were removed under vacuum and the residue suspended in CHCl₃. The precipitate were collected by filtration, washed with MeOH and dried under vacuum. Yield: 189 mg, 63%.

¹H NMR (400 MHz, DMSO-d6): δ 8.98 (s, 1H), 7.97-7.95 (m, 2H), 7.84-7.71 (m, 4H), 7.48-7.37 (m, 3H), 4.07 (s, 1H), 3.31 (s, 6H); ¹³C NMR (100 MHz, DMSO-d6): δ 166.0, 142.8, 140.2, 139.9, 138.6, 133.1, 129.5, 128.5, 127.8, 127.7, 127.1, 126.9, 81.8, 73.3, 29.0; HRMS calculated for C₂₂H₁₈NO [M+H]⁺: 312.1388, found 312.1361.

### Preparation of Boc-Nisin [1-11]Lys(Boc)-OH (Comparative Compound F)

Boc₂O (50 mg, 229 μmol) and DIPEA (51 μL, 293 μmol) were added to a solution of nisin [1-12] (100 mg, 86.9 μmol) in dry MeOH (30 mL) and the mixture was stirred for 4.5 hours. The reaction mixture was concentrated, redissolved in H₂O/MeCN/TFA (70/30/0.1) and purified by preparative HPLC using a C18 Maisch 250×22 mm to yield 68.9 mg (51.0 μmol) of white powder (57% yield). ESI-MS: calcd for C₆₁H₉₉ON₁₃O₁₇S₂ [M+H]⁺ 1350.6796, found 1350.6818.

### Preparation of ((S)-2-amino-N-decyl-3-(1H-indol-3-yl)propanamide)

Prepared via procedure 7 (p7) using Boc-Trp-OH. Yield=88% ¹H NMR (CD₃OD): δ 7.55 (d, 7.88 Hz, 1H), 7.29 (d, 8.12 Hz, 1H), 7.04 (t, 7.50 Hz, 2H), 6.96 (t, 7.42 Hz, 1H), 3.55 (t, 6.72 Hz, 1H), 3.14-2.91 (m, 4H), 1.32-1.00 (m, 16H), 0.90-0.82 (t, 6.64 Hz, 3H). ¹³C NMR (CD₃OD): δ 176.70, 138.1, 128.8, 124.7, 122.4, 119.8, 119.5, 112.3, 111.1, 57.0, 40.4, 33.0, 32.3, 30.5, 30.1, 27.9, 23.7, 18.2, 14.5. ESI-MS: calcd for C₂₁H₃₃N₃O [M+H]⁺ 344.26, found 344.15.

### Preparation of ((S)-2-amino-N-decyl-3-(4-hydroxyphenyl)propanamide)

Prepared via procedure 7 (p7) using Boc-Tyr-OH. Yield=31% ¹H NMR (400 MHz; CD₃OD): δ 8.17-8.10 (m, 1H), 7.04 (d, J=8.0 Hz, 2H), 6.74 (d, J=7.6 Hz, 2H), 3.90 (t, J=7.0 Hz, 1H), 3.25-3.11 (m, 2H), 3.08-2.86 (m, 2H), 1.39-1.36 (m, 2H), 1.35-1.09 (m, 14H), 0.87 (t, J=6.4 Hz, 3H). ¹³C NMR (100 MHz; CD₃OD): 6168.0, 156.8, 130.1, 124.6, 115.3, 54.6, 39.2, 36.6, 31.6, 29.3, 29.2, 29.0, 28.9 28.7, 26.5, 22.3, 13.0. ESI-MS: calcd for C₁₉H₃₂N₂O₂[M+H]⁺: 321.2537, found 321.75.

### Preparation of ((S)-2-amino-N-decyl-3-phenylpropanamide)

Prepared via procedure 7 (p7) using Boc-Phe-OH. Yield=3% ¹H NMR (CD₃OD): δ 7.39-7.23 (m, 5H), 3.99 (t, J=7.48 Hz, 1H), 3.24-3.01 (m, 4H), 1.43-1.13 (m, 16H), 0.90 (t, J=6.86 Hz, 3H). ¹³C NMR (CD₃OD): δ 135.7, 130.5, 130.0, 128.8, 55.9, 40.6, 38.8, 33.1, 30.5, 30.1, 27.9, 23.7, 14.4. ESI-MS: calcd for C₁₉H₃₂N₂O [M+H]⁺ 305.25, found 305.15.

### Preparation of ((S)-2-amino-N-decyl-3-(1H-imidazol-4-yl)propanamide)

Prepared via procedure 7 (p7) using Boc-His-OH. Yield=quant. ¹H NMR (CD₃OD): δ 8.84 (d, J=1.2 Hz, 1H), 8.24 (bs, 1H), 7.07 (bs, 1H), 4.18-4.12 (m, 1H), 3.31-3.27 (m, 2H), 3.18 (t, J=7.2 Hz, 2H), 1.31-1.23 (m, 16H), 0.87 (t, 3H). ¹³C NMR (100 MHz; CD₃OD): δ 143.9, 140.8, 118.1, 52.3, 39.4, 31.6, 29.2, 29.0, 28.9, 28.8, 28.7, 26.5, 22.3, 13.0. ESI-MS: calcd for C₁₆H₃₀N₄O [M+H]⁺: 295.2492, found 295.20.

### Preparation of (tert-butyl ((S)-1-(((S)-1-(decylamino)-3-(1H-indol-3-yl)-1-oxopropan-2-yl)amino)-3-(1H-indol-3-yl)-1-oxopropan-2-yl)carbamate)

A solution of H-Trp-C10 (1.0 g, 2.91 mmol), Boc-Trp-OH (1.1 eq.) BOP (1.1 eq.) and DIPEA (3.3 eq) in CH₂Cl₂ was stirred for 1 hour at room temperature. The reaction mixture was concentrated, taken up in EtOAc and washed with 1M KHSO₄ and sat.NaHCO₃. Drying with Na₂SO₄ and concentrating yielded Boc-Trp-Trp-C10 as yellow solid foam (1.45 g, 2.30 mmol, yield: 79%). This material (0.5 g, 0.79 mmol) was treated with TFA/CH₂Cl₂ in the presence of TiS (0.195 mL, 0.95 mmol) for 1 hour at room temperature. After concentration, the residue was taken up in EtOAc and washed with sat. NaHCO₃. Drying with Na₂SO₄ and concentrating yielded H-Trp-Trp-C10 as oily substance (quantitative yield). MS analysis confirmed removal of the Boc group and the material was used without further purification. Boc-Trp-Trp-C10 ¹H NMR: (CDCl₃): δ 8.73 (s, 1H), 8.43 (1H), 7.63 (d, J=7.6 Hz, 1H), 7.42 (d, J=8.0 Hz, 1H), 7.29-7.00 (m, 6H), 6.73-6.63 (m, 3H), 6.40 (s, 1H), 6.33 (d, J=7.6 Hz, 1H), 4.90 (d, J=5.2 Hz, 1H), 4.66 (m, 1H), 4.26 (m, 1H), 3.45-3.33 (m, 2H), 3.13-3.08 (m, 2H), 2.99-2.96 (m, 1H), 2.58-2.54 (m, 1H), 1.46-1.02 (m, 25H), 0.87 (t, J=6.8 Hz). ¹³C NMR: (100 MHz; CDCl₃): δ 171.9, 171.1, 155.9, 136.7, 136.3, 127.6, 127.4, 123.6, 123.5, 122.8, 22.3, 120.1, 119.7, 119.0, 118.4, 111.7, 111.4, 109.8, 55.7, 53.8, 39.9, 32.0, 29.7, 29.6, 29.4, 29.4, 29.2, 27.9, 26.9, 22.8, 14.2. MS: [M+H]⁺ calcd 630.4014; measured 629.89. H-Trp-Trp-C10: MS [M+H]⁺ calcd 530.3490; measured 530.10.

### Preparation of (2,5-dioxo-2,5-dihydro-1H-pyrrol-1-yl 3-((tert-butoxycarbonyl) amino) propanoate)

DCC (5.5 g, 26.6 mmol) in EtOAc (10 mL) was added to a mixture of Boc-β-Ala-OH (5.0 g, 26.4 mmol) and NHS (3.1 g, 26.9 mmol) in EtOAc (100 mL). The white suspension was stirred overnight at room temperature followed by filtration over celite. The clear filtrate was concentrated and recrystallized from MTBE/hexanes to yield white crystals (6.05 g, 21.1 mmol).

Yield: 80% ¹H NMR: (CDCl₃): δ 5.14 (br, 1H), 3.48-3.46 (m, 2H), 2.81-2.79 (m, 6H), 1.40 (s, 9H). ¹³C NMR: (100 MHz; CDCl₃): δ 169.2, 167.6, 155.8, 79.7, 36.2, 32.2, 28.4, 25.6. MS: [M-tBu+H]⁺ calcd 229.0455; measured 230.71.

### Procedure 1 (p1): Amide Coupled Lipid-Nisin

The resulting powder of the nisin [1-12] structure was dissolved in DMF or THF (240 μl) and the corresponding lipid-amine (59 equivalents), BOP (2 equivalents) and DiPEA (4 equivalents) were added. The reaction was stirred for 20 min and subsequently quenched with 4 mL buffer A (H₂O:MeCN, 95:5+0.1% TFA). The solution was centrifuged for 5 min at 5000 rpm to remove any insoluble material and the supernatant was purified via preparative HPLC. Product fractions were lyophilized to obtain the final product.

### Procedure 2 (p2): Clicked Lipid-Nisin

A 10× stock solution of copper sulfate (16.2 μmol, 2.59 mg in 1 mL H₂O), a 10× stock solution of sodium ascorbate (32.4 μmol, 6.42 mg in 1 mL H₂O), and a 10× stock solution of TBTA (4.1 μmol, 2.18 mg in 1 mL DMF) were prepared. Nisin [1-12]-azide was prepared using procedure 1 (p1) as indicated in Table 3 (Comparative compound E). Nisin [1-12]-azide (8.1 μmol, 10 mg) was dissolved in DMF (200 μL). Lipid-alkyne was added to the μW vessel. The nisin [1-12]-azide solution was added along with 100 μL of the TBTA stock solution, 100 μL of the sodium ascorbate stock solution and 100 μL of the copper sulfate stock solution. The vessel was put in the microwave and reacted at 80° C. for 20 min. After completion, the reaction mixture was quenched with 4 mL buffer B (H₂O:MeCN, 5:95+0.1% TFA) and purified via preparative HPLC.

### Procedure 3 (p3): Lipid-Alkynes

The lipid-amine (3 mmol) was dissolved in DMF (20 mL) and 2,5-dioxopyrrolidin-1-yl pent-4-ynoate (2.0 mmol, 390 mg) was added while stirring and the reaction was allowed to run for 16 hours. After evaporation of DMF the product was purified with flash column chromatography (EtOAc:PE, 1:4) to obtain the final product.

### Procedure 4 (p4): Amine Coupling to Boc-Protected Nisin [1-12]

The lipid-amine (1.2 eq), BOP (1.2 eq) and DiPEA (3 eq) were added to a solution of Boc-Nisin [1-11]Lys(Boc)-OH (1 eq) in dry CH₂Cl₂ (2 μmol/mL). A few drops of DMF aided in the solution of the compounds. The mixture was stirred for 45 min, concentrated and the residue treated with TFA/TiS/H₂O (95/2.5/2.5) for 1 hour and precipitated in MTBE/hexanes (1:1), centrifuged (5 min at 4.500 rpm). The pellet was dissolved in H₂O/t-BuOH (1:1) and lyophilized. The lyophilized powder was dissolved in 4 mL buffer B (H₂O:MeCN, 5:95+0.1% TFA) and purified via preparative HPLC.

Procedure 5 (p5): Lys¹² Acylated Compounds

Nisin [1-12] was dissolved in DMF/THF (1/1) and 4 eq of DiPEA was added. Dropwise addition of a solution of 1 eq. of the carboxylic acid activated ester dissolved in THF resulted in preferred acylation of the lysine side chain. The addition of more than 1 equivalent of activated ester results in the acylation of both the N-terminus and the Lys¹² side chain After 1.5 hours the reaction mixture was concentrated and purified by preparative HPLC using a Maisch Reprospher 100 C8-Aqua, 250 mm×20 mm. The lipid-amine (1.2 eq), BOP (1.2 eq) and DiPEA (3 eq) were added to a solution of the acylated Nisin [1-12] (1 eq) in DMF/THF (2 μmol/mL). The mixture was stirred for 45 min, concentrated, precipitated in MTBE/hexanes (1:1) and centrifuged (5 min at 4.500 rpm). The pellet was dissolved in H₂O/t-BuOH (1:1) and lyophilized. The lyophilized powder was dissolved in 4 mL buffer B (H₂O:MeCN, 5:95+0.1% TFA) and purified via preparative HPLC. Note: the mono and bis β-Ala acylated variants of Nisin [1-12]-C12 were obtained by treating the respective Boc protected precursors with TFA/TIS/H₂O (95/2.5/2.5) followed by precipitation in MTBE/hexanes and preperative HPLC purification as described before.

### Procedure 6 (p6): Amino-Acid-Lipids

A Boc-protected amino acid (Boc-AA-OH) was dissolved in CH₂Cl₂ and cooled at 0° C. EDC (2.5 eq.), HOBT (2.5 eq), decylamine (1.5 eq) and triethylamine (1.5 eq.) were added and the mixture was stirred overnight while warming to room temperature. The reaction mixture was washed with H₂O, 1M NaOH and 1M HCl. Purification via recrystallization (hexanes/EtOAc) or silica gel column chromatography (petroleum ether/EtOAc) yielded the Boc-AA-decylamine intermediates. The Boc-amino acid-decylamine compound was dissolved in CH₂Cl₂ and TiS (2 eq.) and TFA were added to reach a ratio of CH₂Cl₂:TFA (2:1) and the mixture was stirred for 1 hour. The reaction mixture was concentrated and the deprotected amino acid-C10 was optionally taken up in EtOAc and washed with sat. NaHCO₃. Concentrating yielded the lipidated amino acids as oily substances.

The compounds used for further testing were prepared as indicated in Table 3 and 4. In the Tables the procedure used as well as the specific substances used in the procedure are shown.

TABLE 3
Preparation of antimicrobial compounds according to Formula (1)

| | | Z = NHR₁ | Y = NHR₃ | | |
|---|---|---|---|---|---|
| Compound | X₈ | R₁ = | R₃ = | Procedure used | Comments |
| A | Lys | Z = —OH | H | p1 | |
| D | Lys | —CH₃ | H | p1 | with methylamine and using THF as solvent |
| E | Lys | 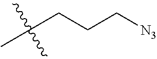 [SMILES: *CCCN=[N+]=[N-]] | H | p1 | With 3-azidopropan-1-amine and using DMF as solvent |
| F | Lys(Boc) | Z = OH | Boc | | |
| (2) | Lys | —C₆H₁₃ | H | p1 | with hexylamine and using DMF as solvent |
| (3) | Lys | —C₇H₁₅ | H | p1 | With heptylamine and using DMF as |
| | | | | | solvent |
| (4) | Lys | —C₈H₁₇ | H | p1 | With octylamine and using DMF as solvent |
| (5) | Lys | —C₉H₁₉ | H | p1 | With nonylamine and using DMF as solvent |
| (6) | Lys | —C₁₀H₂₁ | H | p1 | with decylamine and using DMF as solvent |
| (7) | Lys | —C₁₁H₂₃ | H | p1 | With undecylamine and using DMF as |
| | | | | | solvent |
| (8) | Lys | —C₁₂H₂₅ | H | p1 | With dodecylamine and using DMF as |
| | | | | | solvent |
| (9) | Lys | —C₁₃H₂₇ | H | p1 | With tridecylamine and using DMF as |
| | | | | | solvent |
| (10) | Lys | —C₁₄H₂₉ | H | p1 | with tetradecylamine and using THF as |
| | | | | | solvent |
| (11) | Lys | —C₁₅H₃₁ | H | p1 | With pentadecylamine and using THF as |
| | | | | | solvent |
| (12) | Lys | 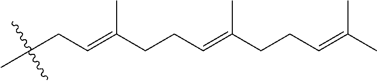 [SMILES: *C/C=C(\C)CC/C=C(\C)CCC=C(C)C] | H | p1 | with farnesylamine and using DMF as solvent |
| (13) | Lys | 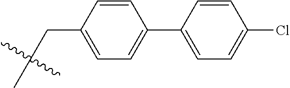 [SMILES: *Cc1ccc(-c2ccc(Cl)cc2)cc1] | H | p4 | With (4′-chloro-[1,1′-biphenyl]-4- yl)methanamine and using CH₂Cl₂ as solvent |
| (14) | Lys | 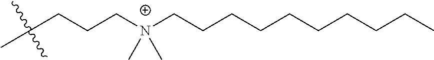 [SMILES: *CCC[N+](C)(C)CCCCCCCCCC] | H | p1 | With N-(3-aminopropyl)-N,N- dimethyldecan-1-aminium 2,2,2- trifluoroacetate and using DMF as solvent |
| (15) | Lys | 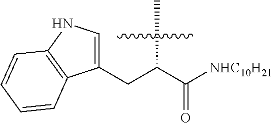 | H | p4 | With (S)-2-amino-N-decyl-3-(1H-indol-3- yl)propanamide and using CH₂Cl₂ as solvent |
| (16) | Lys | 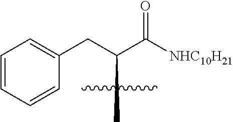 | H | p4 | With (S)-2-amino-N-decyl-3- phenylpropanamide and using CH₂Cl₂ as solvent |
| (17) | Lys | 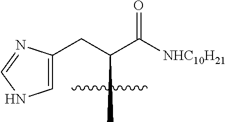 | H | p4 | With (S)-2-amino-N-decyl-3-(1H-imidazol-4- yl)propanamide and using CH₂Cl₂ as solvent |
| (18) | Lys | 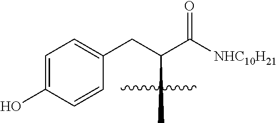 | H | p4 | With (S)-2-amino-N-decyl-3-(4- hydroxyphenyl)propanamide and using CH₂Cl₂ as solvent |
| (19) | Lys((C═O)C₈H₁₇) | —CH₃ | H | p5 | With 2,5-dioxopyrrolidin-1-yl nonanoate, |
| | | | | | methylamine and using DMF as solvent |
| (20) | Acetyllysine | —C₁₂H₂₅ | H | p5 | With 2,5-dioxopyrrolidin-1-yl acetate, |
| | | | | | dodecylamine and using MeOH, then DMF |
| | | | | | as solvents. |
| (21) | 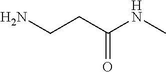 | —C₁₂H₂₅ | H | p5 | With Boc-β-alanine-OSu and dodecylamine, using DMF as solvent |
| (22) | 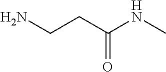 | —C₁₂H₂₅ | 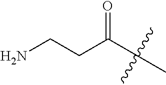 [SMILES: *C(=O)CCN] | p5 | With Boc-β-alanine-OSu and dodecylamine, using DMF as solvent |
| (23) | Lys | 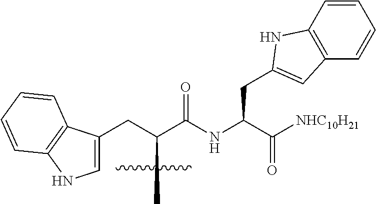 | H | p1 | With (S)-2-amino-N-((S)-1-(decylamino)-3- (1H-indol-3-yl)-1-oxopropan-2-yl)-3-(1H- indol-3-yl)propanamide and using DMF as solvent |

TABLE 4
Preparation of antimicrobial compounds based on Formula (24) with four different R₁ structures.

| R₁ | | | Y = NHR₃ | Procedure | |
|---|---|---|---|---|---|
| structure | X₈ = | R₁ = | R₃ = | used | Comments |
| (b) | Lys | 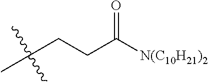 | H | p2 | with didecylalkyne |
| (c) | Lys | 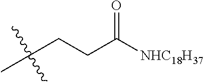 | H | p2 | With octadecyl alkyne |
| (d) | Lys | 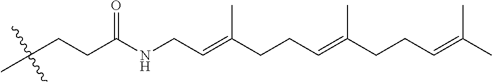 [SMILES: *CCC(=O)NC/C=C(\C)CC/C=C(\C)CCC=C(C)C] | H | p2 | With farnesyl alkyne |
| (e) | Lys | 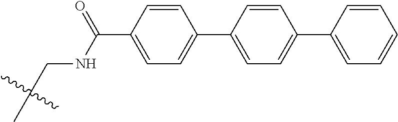 [SMILES: *CNC(=O)c1ccc(-c2ccc(-c3ccccc3)cc2)cc1] | H | p2 | With terphenyl alkyne |

The analysis of the prepared compounds are shown in Tables 5 and 6. The retention times (Rₜ) were measured using a Dr. Maisch C8 column (250×4.6 mm, 300 Å, 10 μm) using a flow rate of 1.0 mL/min and the following gradients: (a) 5-60% MeCN (0.1% TFA) in 40 min; (b) 5-95% MeCN (0.1% TFA) in 40 min, and (c) 5-95% MeCN (0.1% TFA) in 60 min; or using a Dr. Maisch C18 column (250×4.6 mm, 300 Å, 10 μm) using a flow rate of 1.0 mL/min and the following gradients: (d) 5-95% MeCN (0.1% TFA) in 40 min; (e) 5-95% MeCN (0.1% TFA) in 60 min.

TABLE 5
Analysis of the compounds (2) to (23) according to Formula (1), left column. Compounds A, D, E, and F are comparative examples.

| | | Z = NHR₁ | Y = NHR₃ | | Yield | Calculated | Measured | Rₜ |
|---|---|---|---|---|---|---|---|---|
| | X₈ = | R₁ = | R₃ = | Formula | (%) | MW | MW | (min) |
| A | Lys | Z = OH | H | C₅₁H₈₃N₁₃O₁₃S₂ | 37 | 1150.5753 | 1150.5718 | 17.8^{(b)} |
| D | Lys | —CH₃ | H | C₅₂H₈₆N₁₄O₁₂S₂ | 27 | 1163.61 | 1163.63 | 12.2^{(b)} |
| E | Lys | 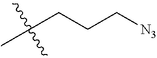 [SMILES: *CCCN=[N+]=[N-]] | H | C₅₄H₈₉N₁₇O₁₂S₂ | 65 | 1232.6396 | 1232.6387 | 18.2^{(a)} |
| F | Lys(Boc) | Z = OH | Boc | C₆₁H₉₉N₁₃O₁₇S₂ | 57 | 1350.6796 | 1350.6818 | 37.93^{(c)} |
| (2) | Lys | —C₆H₁₃ | H | C₅₇H₉₆N₁₄O₁₂S₂ | 30 | 1233.6852 | 1233.6873 | 21.9^{(c)} |
| (3) | Lys | —C₇H₁₅ | H | C₅₈H₉₈N₁₄O₁₂S₂ | 39 | 1247.7003 | 1247.7064 | 30.47^{(e)} |
| (4) | Lys | —C₈H₁₇ | H | C₅₉H₁₀₀N₁₄O₁₂S₂ | 18 | 1261.7159 | 1261.7184 | 31.01^{(e)} |
| (5) | Lys | —C₉H₁₉ | H | C₆₀H₁₀₂N₁₄O₁₂S₂ | 33 | 1275.7316 | 1275.7339 | 32.38^{(e)} |
| (6) | Lys | —C₁₀H₂₁ | H | C₆₁H₁₀₄N₁₄O₁₂S₂ | 45 | 1289.7478 | 1289.7565 | 21.9^{(a)} |
| (7) | Lys | —C₁₁H₂₃ | H | C₆₂H₁₀₆N₁₄O₁₂S₂ | 32 | 1303.7629 | 1303.7623 | 35.17^{(e)} |
| (8) | Lys | —C₁₂H₂₅ | H | C₆₃H₁₀₈N₁₄O₁₂S₂ | 31 | 1317.7785 | 1317.7802 | 36.03^{(e)} |
| (9) | Lys | —C₁₃H₂₇ | H | C₆₄H₁₁₀N₁₄O₁₂S₂ | 26 | 1331.7942 | 1331.7927 | 38.53^{(e)} |
| (10) | Lys | —C₁₄H₂₉ | H | C₆₅H₁₁₂N₁₄O₁₂S₂ | 31 | 1345.8104 | 1345.8109 | 28.4^{(c)} |
| (11) | Lys | —C₁₈H₃₁ | H | C₆₆H₁₁₄N₁₄O₁₂S₂ | 27 | 1359.8255 | 1359.8226 | 42.58^{(e)} |
| (12) | Lys | 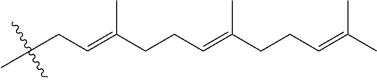 [SMILES: *C/C=C(\C)CC/C=C(\C)CCC=C(C)C] | H | C₆₆H₁₀₈N₁₄O₁₂S₂ | 45 | 1353.7713 | 1353.7821 | 21.5^{(b)} |
| (13) | Lys | 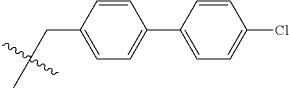 [SMILES: *Cc1ccc(-c2ccc(Cl)cc2)cc1] | H | C₆₄H₉₃ClN₁₄O₁₂S₂ | 23 | 1349.6300 | 1349.6308 | 31.68^{(e)} |
| (14) | Lys | 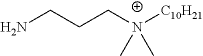 | H | C₆₆H₁₁₆N₁₅O₁₂S₂⁺ | 9 | 1374.8364 | 1374.8357 | 31.20^{(e)} |
| (15) | Lys | 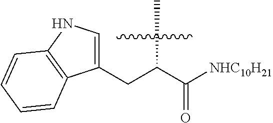 | H | C₇₂H₁₁₄N₁₆O₁₃S₂ | 21 | 1475.8265 | 1474.37 | 36.37^{(e)} |
| (16) | Lys | 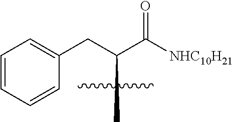 | H | C₇₀H₁₁₃N₁₅O₁₃S₂ | 13 | 1436.8156 | 1437.25 | 36.50^{(e)} |
| (17) | Lys | 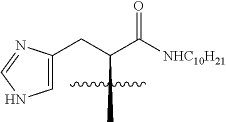 | H | C₆₇H₁₁₁N₁₇O₁₃S₂ | 26 | 1426.8061 | 1426.90 | 31.10^{(e)} |
| (18) | Lys | 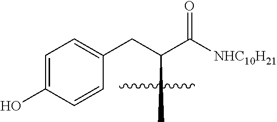 | H | C₇₀H₁₁₃N₁₅O₁₄S₂ | 51 | 1452.8106 | 1452.90 | 34.55^{(e)} |
| (19) | Lys((C═O)C₈H₁₇) | —CH₃ | H | C₆₁H₁₀₂N₁₄O₁₃S₂ | 48 | 1303.7265 | 1304.15 | |
| (20) | Acetyllysine | —C₁₂H₂₅ | H | C₆₅H₁₁₀N₁₄O₁₃S₂ | 36 | 1359.7891 | 1359.65 | 29.87^{(e)} |
| (21) | 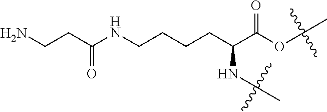 [SMILES: *N[C@@H](CCCCNC(=O)CCN)C(=O)O*] | —C₁₂H₂₅ | H | C₆₆H₁₁₃N₁₅O₁₃S₂ | 6 | 1388.8156 | 1387.98 | 30.03^{(e)} |
| (22) | 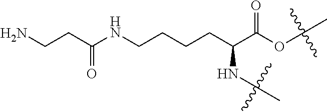 [SMILES: *N[C@@H](CCCCNC(=O)CCN)C(=O)O*] | —C₁₂H₂₅ | 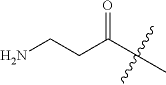 [SMILES: *C(=O)CCN] | C₆₉H₁₁₈N₁₆O₁₄S₂ | 12 | 1459.8528 | 1458.88 | 28.88^{(e)} |
| (23) | Lys | 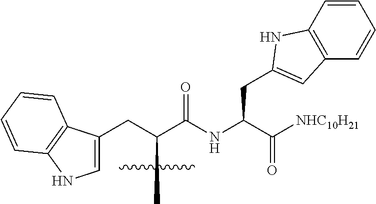 | H | C₈₃H₁₂₄N₁₈O₁₄S₂ | 6 | 1661.9059 | 1663.45 | 38.41^{(e)} |

TABLE 6
Analysis of antimicrobial compounds based on Formula (24) with four different R₁ structures (b), (c), (d) and (e).

| | | | | | | Calculated | Measured | |
|---|---|---|---|---|---|---|---|---|
| struc- | | | Y = NHR₃ | | Yield | MW | MW | Rₜ |
| ture | X₈ = | R₁ = | R₃ = | Formula | (%) | [M + H]⁺ | [M + H]⁺ | (min) |
| (b) | Lys | 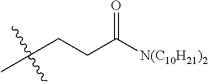 | H | C₇₉H₁₃₆N₁₈O₁₃S₂ | 13 | 1610.0054 | 1610.0011 | 25.2^{(a)} |
| (c) | Lys | 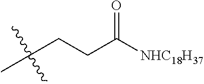 | H | C₇₅H₁₃₂N₁₈O₁₃S₂ | 24 | 1581.9741 | 1581.9741 | 30.9^{(a)} |
| (d) | Lys | 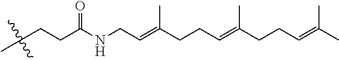 [SMILES: *CCC(=O)NC/C=C(\C)CC/C=C(\C)CCC=C(C)C] | H | C₇₄H₁₂₀N₁₈O₁₃S₂ | 60 | 1533.8802 | 1533.8789 | 19.6^{(b)} |
| (e) | Lys | 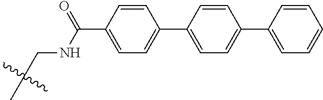 [SMILES: *CNC(=O)c1ccc(-c2ccc(-c3ccccc3)cc2)cc1] | H | C₇₆H₁₀₆N₁₈O₁₃S₂ | 20 | 1543.7706 | 1543.7703 | 22.2^{(a)} |

### Example 2. Analysis of Antimicrobial Compounds in a MIC Assay

Different microorganisms (from glycerol stock) were plated out on blood agar and incubated at 37° C. for 24 hours. A colony was selected and 2×5 mL of TSB was inoculated. The samples and a sterile control were cultured for 16-20 hours at 37° C. Comparative compound B is nisin acting as a control. Comparative compound C is vancomycin, also acting as a control. Comparative compounds A, D and E are also control compounds not part of the present invention.

100 μL of the compound (2) to (23), and compound (24) with R₁-groups (b) to (e), and Comparative compounds A, D and E (128 μg/mL, 2% DMSO in TSB), 100 μL of the positive controls of nisin (Comparative compound B) and vancomycin (Comparative compound C) (2 μg/mL, 2% DMSO in TSB), and 100 μL of a negative control (2% DMSO in TSB), were added to the top of the row of a 96-well plate, 50 μL of TSB to the rest of the wells and the compounds were diluted serially. The overnight cultures were diluted to 0.5×10⁶ CFU in TSB. 50 μL of the bacterial solution was added to each well and the plates were sealed with an adhesive membrane and incubated at 37° C. for 16 hours. The next day, the plates were visually inspected for bacterial growth.

The results of the MIC assays for various bacteria are shown in Table 7. Table 8 shows the activity of compound (10) against a large number of different VRE strains, allowing for the determination of a MIC₅₀ and MIC₉₀, which were 4 and 8, respectively. The same values were found for comparative compound B (nisin), illustrating the potency of the new compounds.

TABLE 7
MIC values^{a} (measured in μg/mL). All data stem from duplicate
experiments. Where appropriate, values are represented as a range.

| | | | | | | MRSA | MRSA |
|---|---|---|---|---|---|---|---|
| Compound | B. subtilis | S. aureus | E. coli | M. luteus | VRE 155 | WKZ2 | USA300 |
| A | >128 | >128 | >128 | 8-16 | >128 | >128 | >128 |
| B | 0.625-2.5 | 10 | >10 | 0.02-0.03 | 5 | 10 | 10 |
| C | 0.03-0.06 | 0.25-0.31 | >10 | 0.03 | >10 | 0.625 | 0.625 |
| D | >128 | >128 | >128 | — | — | — | — |
| E | >128 | >128 | >128 | — | — | — | — |
| (2) | 32 | >64 | >64 | 2 | — | — | — |
| (3) | 16 | 64 | >64 | — | 8 | — | 64 |
| (4) | — | — | — | — | 16 | — | 64 |
| (5) | 8 | 16 | 64 | — | 8-16 | — | 32 |
| (6) | 4-8 | 16 | 64 | 2 | 8 | 16 | 16 |
| (7) | 4 | 8 | >64 | — | 4 | — | 16 |
| (8) | 4 | 4-8 | >64 | — | 2 | — | 4 |
| (9) | 4 | 8 | >64 | — | 2 | — | 4 |
| (10) | 4 | 16 | >64 | 1-2 | 4 | 32 | 64 |
| (11) | 8 | 32 | >64 | — | 8 | — | 64 |
| (12) | 4 | 8 | >64 | 2 | 8 | 16 | 16 |
| (13) | 2-4 | — | — | — | 4 | — | 8 |
| (14) | 4 | 4 | >64 | — | 4 | — | 8 |
| (15) | 4 | 8 | >64 | — | 2 | — | 8 |
| (16) | 4 | 8 | >64 | — | 2 | — | 8 |
| (17) | — | — | — | — | 4 | — | 8 |
| (18) | 4 | 4-8 | >64 | — | 2 | — | 8 |
| (19) | — | — | — | — | 8 | — | 32 |
| (20) | — | — | — | — | 4 | — | >64 |
| (21) | — | — | — | — | 4 | — | 8 |
| (22) | — | — | — | — | 8 | — | 16 |
| (23) | — | — | — | — | 4-8 | — | 16 |
| (24) with | >64 | >64 | >64 | 8 | — | — | — |
| R₁₋group | | | | | | | |
| (b) | | | | | | | |
| (24) with | >64 | >64 | >64 | 8 | — | — | — |
| R₁₋group | | | | | | | |
| (c) | | | | | | | |
| (24) with | 32 | 64 | >64 | 2-4 | — | — | — |
| R₁₋group | | | | | | | |
| (d) | | | | | | | |
| (24) with | 4 | 8 | >64 | 2 | 8 | 8 | 8 |
| R₁₋group | | | | | | | |
| (e) | | | | | | | |

TABLE 8
MIC₅₀ and MIC₉₀ determination for compound (10) and
Comparative compounds B and C against thirty VRE strains
(MIC measured in μg/mL). All data stems from duplicate experiments.
Where appropriate, values are represented as a range.

| | | | | Comparative | Comparative |
|---|---|---|---|---|---|
| Strain | | van | compound | compound B | compound C |
| ID^{a} | Country | gene | (10) | (nisin) | (vancomycin) |
| E0013 | GBR | vanA | 4 | 8 | 32 |
| E0072 | NLD | vanA | 2 | 2 | 8 |
| E0300 | USA | vanA | 8-16 | 8 | >128 |
| E0321 | FRA | vanA | 2 | 2 | >128 |
| E0333 | ISR | vanA | 4 | 4-8 | >128 |
| E0338 | ITA | vanA | 4 | 8 | >128 |
| E0341 | GBR | vanA | 4 | 4 | 128 |
| E0506 | AUS | vanA | 8 | 4-8 | >128 |
| E0745 | NLD | vanA | 4 | 4 | 64 |
| E1130 | USA | vanA | 4 | 4-8 | >128 |
| E1441 | GRC | vanA | 4 | 4 | 128 |
| E1679 | BRA | vanA | 4 | 4 | >128 |
| E1763 | BEL | vanA | 8 | 8 | >128 |
| E2297 | USA | vanA | 2 | 4 | 128 |
| E2359 | SGP | vanB | 2 | 8 | <1-1 |
| E2365 | HUN | vanB | 1 | 2-4 | <1 |
| E2373 | SRB | vanA | 4 | 4 | >128 |
| E6016 | LVA | vanA | 2-4 | 4 | 128 |
| E7312 | NLD | vanA | 2 | 4 | 64 |
| E7314 | NLD | vanB | 4 | 4-8 | >128 |
| E7319 | NLD | vanA | 2 | 4 | >128 |
| E7329 | NLD | vanA | 2 | 4 | <1 |
| E7401 | NLD | vanB | 4 | 8 | 16 |
| E7403 | NLD | vanB | 1 | 4 | <1 |
| E7413 | NLD | vanA | 4 | 4 | >128 |
| E7424 | NLD | vanB | 1-2 | 4 | >128 |
| E7464 | NLD | vanB | 4 | 4 | 2 |
| E8218 | NLD | vanB | 2 | 4 | 8 |
| E8235 | NLD | vanB | 4 | 4 | 8 |
| E8237 | NLD | vanA | 8 | 8 | 128 |
| | | | MIC₉₀ = 8 | MIC₉₀ = 8 | MIC₉₀ = >128 |
| | | | MIC₅₀ = 4 | MIC₅₀ = 4 | MIC₅₀ = 128 |

^{a}See http://www.nationsonline.org/oneworld/countrycodes.htm for country codes.

### Example 3. Lipid II-Binding in Model Membranes

Large unilamellar vesicles (LUVs), composed of 1,2-dioleoyl-sn-glycero-3-phosphocholine (DOPC) spiked with 0.2% lipid II, were loaded with carboxyfluorescein (CF). The CF efflux was monitored by measuring the increase in fluorescence intensity at 515 nm, with excitation at 492 nm. In a cuvette, a solution (1 mL) of CF-loaded vesicles (20 μM final concentration) in buffer (Tris.HCl, pH 7.0 containing 100 mM NaCl) was prepared and the relevant final concentration of compounds (6), (10), and (12), and also compound (24) with R₁-group (e), were added and the mixture was stirred for 1 min and the fluorescence was recorded (A₀). After ca. 10 seconds, nisin (comparative compound B) was added (5 nM final concentration) and the fluorescence was followed until it stabilized, then recorded (A_{stable}). Total membrane leakage was induced by the addition of Triton-X100 (final concentration 0.1%) and the fluorescence was recorded (Aₜₒₜₐₗ). The percentile values were calculated by:

$$\frac{{A}_{stable} - {A}_{0}}{{A}_{total} - {A}_{0}} \times 100 \% .$$

Treatment with the compounds (6), (10), and (12) and treatment with compound (24) with R₁-group (e) did not show any detectable dye leakage, whereas nisin (comparative compound B) at a concentration of 5 nM resulted in leakage of about 50% of the CF dye. The competition assay revealed that each compound effectively antagonized nisin induced membrane leakage when administered at a concentration 10-fold higher than nisin, except for compound 24 with R₁-group e, which inhibited dye leakage at equimolar concentrations, suggesting a binding affinity for lipid II on par with nisin in this model.

### Example 4. Serum Stability

2 mg/mL peptide solutions were prepared in 26% DMSO in MilliQ. Duplicate samples were prepared with 42 mL peptide solution and 518 mL human serum, making the final DMSO concentration 2%. The samples were incubated at 37° C., and samples were taken at t=0, 1, 2, 4 and 24 hours as follows: to 100 μL serum solution, 200 μL MeOH (containing 0.075 mg/mL ethylparaben as an internal standard) was added to precipitate the proteins. The sample was vortexed briefly and allowed to stand for 10 min at RT. The samples were then centrifuged at 13,000 rpm for 5 min, and the supernatant was taken and stored at −20° C. until analysis. Each sample was analyzed by HPLC on a C4 column. The peaks were integrated and normalized to the internal standard.

The stability of compounds (6), (10), and (12) and the stability of compound (24) with R₁-group (e) in human serum was compared to the stability of nisin [1-12] (compound B). It was found that the stability of the new compounds was well above 50%, and significantly exceeded the stability of compound B, i.e. only 33% of nisin remained intact after 24 hours, whereas 94% of compound (6) remained intact after 24 hours.

### Example 5. Hemolysis Assay

Human whole blood was centrifuged at 600×g for 15 min and levels of plasma and hematocrit were marked on the tube. Plasma was removed and the erythrocytes washed 3× with PBS (centrifuging at 600×g for 15 mins). After discarding the supernatant, the packed cells were stored on ice. 100 μL of the peptides (128 μg/mL in PBS, 2% DMSO) as well as a control solution comprised of 2% DMSO in PBS were added to the top row of a polypropylene, round-bottom 96 well plate and 50 μL of PBS to the rest of the wells. The peptides and the DMSO control solutions were then diluted serially down the rows. 200 μL of the packed cells were added to PBS (10 mL) and 50 μL of this suspension was added to each well. A column with DI water containing 0.1% Triton X-100 was used as the 100% lysis control, and the column containing the serially-diluted PBS (1.0% DMSO) control served as the 0% lysis reference. The cells were incubated at 37° C. for 1 h. After incubation the plates were centrifuged (800×g, 5 min) and 25 μL of the supernatant was added to 100 μL DI water in a flat-bottom plate (polystyrene). The absorption at 414 nm was recorded to measure the amount of free hemoglobin. The hemolysis of compounds (6), (10), (12), and (20) and the hemolysis of compound (24) carrying R₁-group (e) was compared to Comparative compounds B and C. All new compounds showed a level of hemolysis below 15% at concentrations as high as 32 μg/mL. Comparative compounds B and C show negligible hemolysis at 32 μg/mL. Compound (20) showed no detectable hemolysis up to the highest concentration tested (64 μg/mL), which illustrates the preferable masking of the positive charge on the Lys¹² group to prevent hemolysis.

### Example 6. BioScreen Growth Assays with E. faecium

A BioScreen C instrument (Oy Growth Curves AB, Helsinki, Finland) was used to monitor effects of the compounds (6), (10), and (12) and the effect of compound (24) with R₁-group (e), or Comparative compound B (nisin) on E. faecium growth (each compound administered at a fixed concentration 5 μM). E. faecium strains were inoculated at an initial OD₆₆₀ of 0.05 into 300 μl TSB containing 1% DMSO and 1% glucose or into the same medium containing the antibiotic compounds at a final concentration of 5 μM. The cultures were incubated in the Bioscreen C system at 37° C. with continuous shaking, and the absorbance at 600 nm (A₆₀₀) recorded every 15 min for 15 hours to determine growth/inhibitory effects.

The E. faecium strains used in these experiments are E. faecium E745 (vancomycin-ampicillin resistant hospital outbreak strain), E. faecium E980 (vancomycin-ampicillin susceptible human commensal isolate), E. Faecium E1133 (vancomycin-ampicillin resistant hospital outbreak strain), and E. faecium E1162 (vancomycin-susceptible ampicillin-resistant clinical isolate). The BioScreen growth assays for each strain was determined for the compounds (6), (10), (12) and also for compound (24) with R₁-group (e) as well as nisin (Comparative compound B) and compared to non-treated strains, the results being shown in the Tables 9 to 12 below.

TABLE 9
BioScreen growth assay for E. faecium E745.

| | OD₆₀₀ | | | | | |
|---|---|---|---|---|---|---|
| | | | | | | compound |
| Time | No | compound B | compound | compound | compound | (24) with R₁- |
| (h) | treatment | (nisin) | (6) | (10) | (12) | group (e) |
| 0 | 0.013667 | 0.017333 | 0.012000 | 0.015333 | 0.015333 | 0.015000 |
| 4 | 0.434333 | 0.010333 | 0.069333 | 0.012000 | 0.009000 | 0.013667 |
| 8 | 0.835000 | 0.010333 | 0.575667 | 0.011667 | 0.008667 | 0.017667 |
| 12 | 0.818333 | 0.010667 | 0.685333 | 0.011667 | 0.009667 | 0.022667 |
| 16 | 0.801667 | 0.008667 | 0.679000 | 0.011333 | 0.010000 | 0.031000 |

TABLE 10
BioScreen growth assay for E. faecium E980.

| | OD₆₀₀ | | | | | |
|---|---|---|---|---|---|---|
| | | | | | | compound |
| Time | No | compound B | compound | compound | compound | (24) with R₁- |
| (h) | treatment | (nisin) | (6) | (10) | (12) | group (e) |
| 0 | 0.016333 | 0.016000 | 0.017333 | 0.018333 | 0.017333 | 0.017333 |
| 4 | 0.831667 | 0.011667 | 0.464000 | 0.015000 | 0.012000 | 0.022333 |
| 8 | 0.955667 | 0.011000 | 0.913667 | 0.014333 | 0.013000 | 0.072000 |
| 12 | 0.893000 | 0.011000 | 0.886000 | 0.013667 | 0.016667 | 0.269667 |
| 16 | 0.802333 | 0.010667 | 0.849667 | 0.013667 | 0.027333 | 0.366667 |

TABLE 11
BioScreen growth assay for E. faecium E1133

| | OD₆₀₀ | | | | | |
|---|---|---|---|---|---|---|
| | | | | | | compound |
| Time | No | compound B | compound | compound | compound | (24) with R₁- |
| (h) | treatment | (nisin) | (6) | (10) | (12) | group (e) |
| 0 | 0.014667 | 0.016000 | 0.016000 | 0.017000 | 0.016667 | 0.016667 |
| 4 | 0.596000 | 0.009667 | 0.226667 | 0.012667 | 0.010333 | 0.024667 |
| 8 | 0.703667 | 0.009333 | 0.666333 | 0.011667 | 0.013667 | 0.102333 |
| 12 | 0.689333 | 0.009333 | 0.665333 | 0.011667 | 0.026667 | 0.403000 |
| 16 | 0.676000 | 0.008667 | 0.629000 | 0.011000 | 0.124333 | 0.514000 |

TABLE 12
BioScreen growth assay for E. faecium E1162.

| | OD₆₀₀ | | | | | |
|---|---|---|---|---|---|---|
| | | | | | | compound |
| Time | No | compound B | compound | compound | compound | (24) with R₁- |
| (h) | treatment | (nisin) | (6) | (10) | (12) | group (e) |
| 0 | 0.015667 | 0.024000 | 0.013667 | 0.019000 | 0.017000 | 0.018667 |
| 4 | 0.719333 | 0.015333 | 0.194000 | 0.013667 | 0.011000 | 0.027333 |
| 8 | 0.842667 | 0.011333 | 0.671333 | 0.013000 | 0.012333 | 0.095333 |
| 12 | 0.827667 | 0.012000 | 0.678667 | 0.012667 | 0.016667 | 0.378333 |
| 16 | 0.814333 | 0.010333 | 0.665667 | 0.012333 | 0.025667 | 0.397000 |

The conclusion is that all compounds that were generated and produced in accordance with the present invention as well as nisin demonstrate a delay as well as an inhibition of the growth of all tested E. faecium strains. The compounds (6) and (10) and also compound with Formula (24) carrying R₁-group (e), and in particular compound (12) showed very good growth inhibition performances.

1. An antimicrobial compound according to Formula (1), 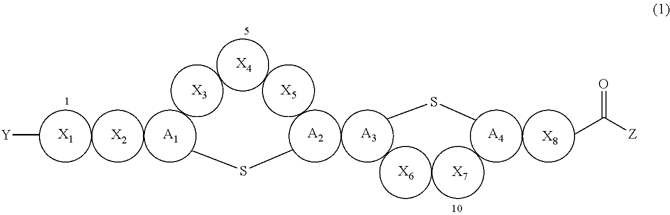
wherein:
Z is selected from any one of the substituents NHR₁, NR₁R₂, OR₁ and SR₁, and Y is selected from any one of the substituents NHR₃, NR₃R₄, NHCR₃R₄, NHCOR₃, NHCSR₃, NHOR₃ and NHC(NR₃NHR₄), wherein R₁, R₂, R₃ and/or R₄ is a substituted or non-substituted substituent selected from: alkyl, alkenyl, alkynyl, cycloalkyl, aryl, and polyaryl, wherein said substituent comprises at least 2, 4 or 6 carbon atoms and at most 30, 40 or 50 carbon atoms;
A₁ and A₃ are independently D-Alanine or D-Aminobutyric acid;
A₂ and A₄ are L-alanine;
A₁+A₂ and A₃+A₄ independently form a (2S,6R)-lanthionine or (2S,3S,6R)-methyllanthionine linkage; and
X₁ to X₈ are each independently selected from natural or non-natural amino acids.

2. Antimicrobial compound according to claim 1, wherein Y and Z each have a molecular weight of less than 1200 Dalton.

3. Antimicrobial compound according to claim 1, wherein R₁, R₂, R₃ and/or R₄ is a substituted or unsubstituted substituent, independently selected from: C₄ to C₅₀ alkyl, C₂ to C₅₀ alkenyl, C₂ to C₅₀ alkynyl, cycloalkyl, aryl and polyaryl.

4. Antimicrobial compound according to claim 1, wherein both R₁ and R₂ and/or both R₃ and R₄ are substituted or non-substituted substituents selected from: C₅ to C₄₀ alkyl, C₄ to C₄₀ alkenyl, C₄ to C₄₀ alkynyl, cycloalkyl, aryl and polyaryl.

5. Antimicrobial compound according to claim 1, wherein X₈ is an amino acid that carries no charge on the side-chain.

6. Antimicrobial compound according to claim 5, wherein X₈ is lysine that is acylated or acetylated on the side-chain.

7. Antimicrobial compound according to claim 1, wherein Z has a molecular weight of less than 1000 Dalton, preferably less than 800 Dalton, and more preferably less than 600 Dalton; and/or Y has a molecular weight of less than 1000 Dalton, preferably less than 800 Dalton, and more preferably less than 600 Dalton.

8. Antimicrobial compound according to claim 1, wherein Y is not NH₂, and Z is not OH or NH—CH₃.

9. Antimicrobial compound according to claim 1, wherein said compound has an antimicrobial activity exceeding the activity of the unsubstituted nisin [1-12] structure.

10. Antimicrobial compound according to claim 1, wherein the amide-form of Z independently lacks antimicrobial activity.

11. Antimicrobial compound according to claim 1, wherein the compound exhibits an MIC value below 100 μg/ml, preferably below 70 μg/ml, 50 μg/ml, 20 μg/ml, and most preferably below 10 μg/ml.

12. Antimicrobial compound according to claim 1, for use in the treatment of a bacterial infection.

13. A pharmaceutical composition comprising the antimicrobial compound according to claim 1, and a pharmaceutically acceptable diluent and/or carrier.

14. Use of an antimicrobial compound according to claim 1, for the manufacture of a medicament, for use in the treatment of a bacterial infection.

15. A method of treating a subject suffering from a bacterial infection, comprising administering an antimicrobial compound according to claim 1, to said subject.
